(19) United States
(12) Patent Application Publication
(10) Pub. No.: US 2003/0092628 A1
(43) Pub. Date: May 15, 2003

(54) Gonadotropin releasing hormone antagonist

(76) Inventors: Henrik Johan Matthieu Maria de Greef, BH Utrecht (NL); Bernadette Maria Julia Louise Mannaerts, Heesch (NL); Everardus Otto Maria Orlemans, Long Valley, NY (US); Gerrit G. Voortman, GJ Warnsveld (NL); Herman Jan Tijmen Coelingh Bennink, BE Driebergen (NL)

Correspondence Address: INTERVET INC, 405 STATE STREET, PO BOX 318, MILLSBORO, DE, 19966, US

(21) Appl. No.: 10/319,884

(22) Filed: Dec. 13, 2002

Related U.S. Application Data
(63) Continuation of application No. 09/763,434, filed on Apr. 3, 2001.
(63) Continuation-in-part of application No. 09/446,324, filed on Dec. 20, 1999.

(30) Foreign Application Priority Data
Jun. 23, 1999 (EP) 99202027.1

(51) Int. Cl. A61K 38/24

(52) U.S. Cl. 514/12

(57) ABSTRACT

The present invention relates to a method to prevent a premature LH surge. The method employs the administration of the gonadotropin releasing hormone antagonist ganirelix in an amount dependent on the body weight of the patient. The method can be used in combination with administration of exogenous FSH in the treatment of women undergoing controlled ovarian super-ovulation.

The present invention relates to the use of GnRH antagonists in controlled ovarian hyperstimulation (COH) as well as to a method to prevent premature LH surge. It also relates to a cartridge comprising said antagonist and a kit comprising said cartridge and FSH.

The glycoprotein hormones Luteinizing Hormone (LH) and Follicle Stimulating Hormone (FSH) are released from the pituitary gland under control of Gonadotropin Releasing Hormone (GnRH). They act on the ovary to stimulate steroid synthesis and secretion and thus play a central role in the reproductive cycle.

In the normal cycle, there is a mid-cycle surge in LH concentration which is followed by ovulation. The LH surge is a consequence of the raise in estrogen levels brought about by the endogenous secretion of LH and FSH. The estrogen is part of a positive feedback mechanism resulting in the elevated LH level.

GnRH analogues are useful for a variety of disorders in which immediate reversible suppression of the pituitary-gonadal axis is desired. This can in principle be achieved with GnRH agonists as well as with GnRH antagonists. In comparison to GnRH agonists, GnRH antagonists have the advantage of not inducing an initial release of gonadotropins (flare-up) and steroids before suppression.

Currently, GnRH agonists are clinically applied for the prevention of endogenous LH-surges during controlled ovarian hyperstimulation for Assisted Reproduction Techniques (ART). Specific disadvantages of GnRH agonists are the initial flare-up and the rather long period until pituitary suppression becomes effective. Usually, patients undergoing COH start only treatment with (recombinant) FSH after 2 to 3 weeks pretreatment with GnRH agonists.

Women treated for this purpose without GnRH analogues, all show attenuated LH rises irrespective of the treatment schedule used. Usually these rises occur prematurely due to a positive feedback of rising estradiol (E2) produced by a cohort of relative small follicles. The exposure of non-mature follicles to high levels of LH leads to premature luteinisation of granulosa cells and hence to increased production of progesterone and decreased synthesis of E2. These changes lead to disrupted maturation and decreased fertilization and implantation rates. Success rates of COH cycles in which premature LH rises are detected, are reported to be low and often these cycles are canceled because the number and/or size of follicles is still too small.

GnRH antagonists by GnRH receptor competition provide an immediate inhibition of gonadotropin secretion, especially of LH. Thus, during COH by FSH, GnRH antagonist treatment is only required during the few days when there is an increased risk for a premature LH surge. It has been found that the GnRH antagonist dosage range is critical: too low a GnRH antagonist dosage leading to prmature LH rises, while too high a GnRH antagonist dosage hampered follicular maturation. For the antagonist ganirelix for example a fixed amount being at least 0.125 mg but less than 1 mg and preferably about 0.25 mg was suggested (WO98/58657).

Surprisingly, however, it has now been found that there is no relationship between the implantation rate and level of LH (AUC), whereas there does exist a relationship between the GnRH antagonist levels (AUC) and the implantation rate. It has now been found that antagonist is to be administered in an amount depending on the body weight (BW).

The invention therefore relates to a pharmaceutical preparation comprising GnRH antagonist, while applying a dosage adjusted for body weight sufficient to prevent a premature LH surge and ensuring successful treatment outcome. Such preparation is useful in the treatment of women undergoing COH.

A preferred antagonist according to the present invention is ganirelix which has the following chemical name:

N-Acetyl-3-(2-naphtyl)-D-alanyl-4-chloro-D-phenylalanyl-3-(3-pyridyl)-D-alanyl-L-seryl-L-N⁹,N¹⁰-diethyl-D-homoarginyl-L-leucin-N⁹,N¹⁰-diethyl-L-homoarginyl-L-propyl-D-alanylamide acetate. The abbreviated structure is [N-Ac-D-Na(2)¹, D-pClPhe²,D-Pal(3)³,D-hArg(Et₂)⁶, L-hArg(Et₂)⁸, D-Ala¹⁰-GnRH.

The GnRH antagonist ganirelix is disclosed in U.S. Pat. No. 4,801,577 for nonapeptide and decapeptide analogs of LHRH useful as LHRH antagonists. This patent, which is fully incorporated herein by reference, describes the method for the preparation of these compounds. It is indicated that the compounds described therein can be used for the prevention of ovarian hyperstimulation. For human therapy a daily range is suggested for administration of the active ingredient between 0.001 and 5 mg/kg body weight, preferably between 0.01 and 1 mg/kg.

It has now been found that the optimal relationship between body weight and GnRH antagonist dosage can be defined (in micrograms) by the Formula: (5.5*BW−166)±7% (Formula I) wherein BW represents the body weight of the patient in kg.

The preparation is administered together with FSH during the days of ovarian stimulation when a premature LH rise may easily occur e.g. from day 5 of FSH administration onwards. The preparation in its proposed dosage range has the advantage of providing an immediate effect that prevents an LH surge and at the same time maximizes the chances of establishing pregnancy. Administration is usually stopped when sufficient follicles have matured and exogenous hCG/LH is given for induction of ovulation. The amount of hCG/LH usually amounts 5000-10000 IU. Alternatively, induction of ovulation can be performed by administration of a GnRH agonist. The agonist instead of hCG/LH is usually given on the same day in an amount sufficient to trigger ovulation. A suitable range is 10-1000 μg. Suitable agonists are e.g. buserelin, triptorelin and luprorelin.

The exact regimen for administration might depend on the individual response and is finally to be decided by the clinician who treats the subject. For this reason the duration of initial ovarian stimulation with FSH alone as well as the duration of combined treatment with FSH/GnRH antagonist treatment may vary. FSH treatment usually starts at menses day 1, 2 or 3. Ovarian stimulation with FSH alone may be continued up to 5 days in an amount of e.g. 150-225 IU. FSH is administered preferably as a recombinant protein. Treatment with GnRH antagonist may be started at the first day of FSH, but preferably such treatment starts at FSH treatment day 4 or 5. The GnRH antagonist is administered in the previously determined amount according to the invention in combination with FSH in amounts between 50-600 IU, preferably between 100-300 IU. GnRH antagonist treatment may last 2-14 days i.e. up to the moment whereupon the patient is treated with exogenous LH/hCG or a GnRH or GnRH agonist for ovulation induction.

According to another aspect of the invention ganirelix in an amount according to Formula I is used for the manufacture of a medicament to prevent a premature LH surge in women undergoing controlled ovarian hyperstimulation.

The pharmaceutical preparations for use according to the invention can be prepared in accordance with standard techniques such as for example are described in the standard reference, Gennaro et al. (Ed.), Remmington's Pharmaceutical Sciences, (18^{th} ed. Mack Publishing Company, 1990, e.g. Part 8: Pharmaceutical Preparations And Their Manufacture). For the purpose of making the pharmaceutical preparations according to the invention, the active substance is mixed with or dissolved in a pharmaceutical acceptable carrier.

Any conventional pharmaceutical carrier that does not interfere with performance of the active ingredient can be used in the preparations according to the present invention. Formulations may contain as common excipients sterile water or saline, alkylene glycols such as propylene glycol, polyalkylene glycols such as polyethylene glycol, oils of vegetable origin, hydrogenated naphtalenes and the like.

The pharmaceutical preparation of the antagonist may be administered parenterally. Preferably it is administered subcutaneously, particularly in the form of liquid solutions or suspensions. A typical formulation is a solution containing, in addition to the active substance in an amount as indicated above, glacial acetic acid, mannitol, and water adjusted to pH 5 with sodium hydroxide and/or hydrochloric acid. Optionally preservations such as e.g. methyl- and propylparaben or benzylalcohol can be added. The solutions can be packaged e.g. in glass vials, cartridges or in syringes.

In a further aspect of the invention there is provided a cartridge containing a sterile liquid formulation of ganirelix. As used herein a cartridge means a closed container, such as an ampoule, a vial, a bottle or a bag comprising an amount of GnRH antagonist so as to administer accurately and preferably repeatedly to a patient a dosage of GnRH antagonist according to Formula I.

Thus, a cartridge may contain an amount of the liquid antagonist formulation corresponding to one or more therapeutic dosages of the antagonist. Preferably these dosages are to be applied in a single regimen. Preferably the cartridges contain an amount of GnRH antagonist sufficient for 5 administrations. The cartridges are preferably used in combination with a device making it possible to deliver adjustable dosages needed in the regimen.

In another aspect of the invention there is provided a device for administration comprising a cartridge containing a sterile liquid formulation according to the invention. A preferred device for administration is a pen-type injector, which comprise means for easy adjustment of the amount of a formulation that is to be injected. Such pen type injectors are known per se, such as for instance the well known B-D Pen (a trademark of Becton Dickinson and Company), an insulin-injection system.

Adjustable cartridges according to the invention have the advantage of accurate self-administration thereby increasing the convenience for the patients.

In yet another aspect of the invention there is provided a kit for use in controlled ovarian hyperstimulation in female patients. Such a kit comprises a GnRH antagonist in a dosage form and quantity so as to accurately administer to a patient in an amount according to Formula I, in a frequency effective to prevent a premature LH surge. In addition the kit comprises FSH in a dosage form and quantity suitable for administering in an amount and frequency effective to stimulate growth of follicles. Optionally the kit may comprise also hCG/LH or GnRH agonists in a dosage form and quantity suitable for administering in an amount and frequency effective to induce ovulation. The GnRH antagonist preferably is packaged in a cartridge. This cartridge preferably is to be used in combination with a device for administration such as a pen type injector allowing an adjustable and accurate administration of GnRH antagonist. Thus, the kit might also comprise a pen type injector system.

The invention is further explained by reference to the following Examples.

### LEGENDS TO THE FIGURES

FIG. 1: LH levels were measured at the start Oust before the first injection of ganirelix) and end of ganirelix treatment. The graph shows the pregnancies in relation to the various levels. LH levels are indicated in IU/L.

FIG. 2: Area under the curve of ganirelix versus body weight of subjects in three pharmacokinetic studies. Circles mean protocol A; triangles mean protocol B and squares mean protocol C.

FIG. 3: Chance of pregnancy versus body weight; results from Phase III efficacy study. Dots in the top of the graph correspond to pregnant subjects (100%), while for subjects not pregnant (0%) these dots are displayed at the bottom of the graph. The spline function (chance of pregnancy vs body weight) is the resulting curve of these observations.

### EXAMPLES

### Example 1

LH Serum Levels Versus Pregnancy

Recombinant FSH (recFSH) treatment was started on day 2 or 3 of the menstrual cycle by a once daily SC injection. Just prior to the first injection of recFSH an hCG test was performed to exclude pregnancy, a blood sample for hormone analysis was taken and an ultrasonography (USS) was performed. During recFSH treatment day 1 through 5, the daily dose of recFSH was fixed to 150 international units (IU). On day 6 of recFSH treatment ganirelix treatment was started by daily SC administration until and including the day before the day of hCG.

During ganirelix treatment, the dose of recFSH was adjusted depending on the individual ovarian response as assessed by USS. From the first day of ganirelix i.e. from recFSH treatment day 6 onwards up to and including the day of hCG, a blood sample for hormone analysis was taken prior to drug administration. And an USS was performed, at least every two days.

LH levels were assessed by a standard LH specific assay at the Central Laboratory of the Analytisch Biochemisch Laboratorium (Assen, The Netherlands). From the data on LH levels, a plot was constructed in order to investigate the possible role of LH on pregnancy.

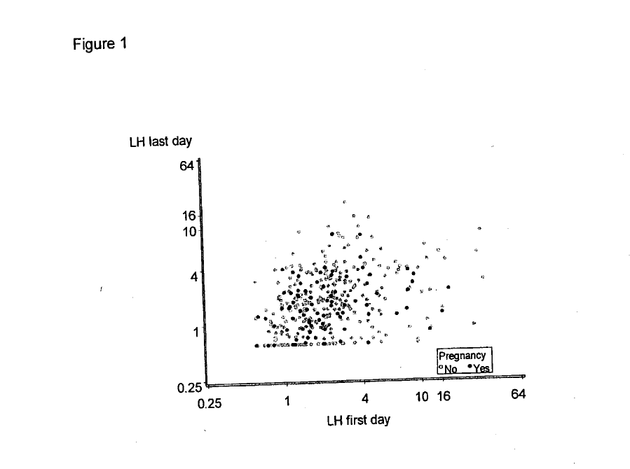

LH levels were measured at the start (just before the first injection of ganirelix) and end of ganirelix treatment. FIG. 1 shows the pregnancies in relation to the various levels.

Clearly, from this graph, no relationship between LH serum levels and pregnancy outcome was found.

### Example 2

Body Weight Versus Pregnancy

For the body weight parameter the treated groups of example 1 were divided into categories of a 10 kg range and the pregnancy rates found for these separate categories were investigated. The results are indicated in Table 1

TABLE 1
Relationship between pregnancy and body weight

| Weight (kg) | Pregnancy rate |
|---|---|
| <50 | 9% |
| 50-60 | 16% |
| 60-70 | 22% |
| 70-80 | 22% |
| >=80 | 22% |

From this table a relationship between the parameter body weight and clinical outcome (i.e. pregnancy) was observed.

### Example 3

Body Weight vs AUC

Several pharmacokinetic studies were carried out with ganirelix. Protocol A: an open-label two-way crossover study to assess the absolute bioavailablity of 0.25 mg ganirelix after single injection. Protocol B: an open-label randomized, multiple dose parallel-design study to assess the dose-proportionality and the pharmacokinetic properties of ganirelix (0.125, 0.25 and 0.5 mg) after repeated subcutaneous administration. Protocol C: an open, randomized, two-way crossover study to establish the local tolerance and bioavailability of ganirelix after multiple subcutaneous administration (2 mg).

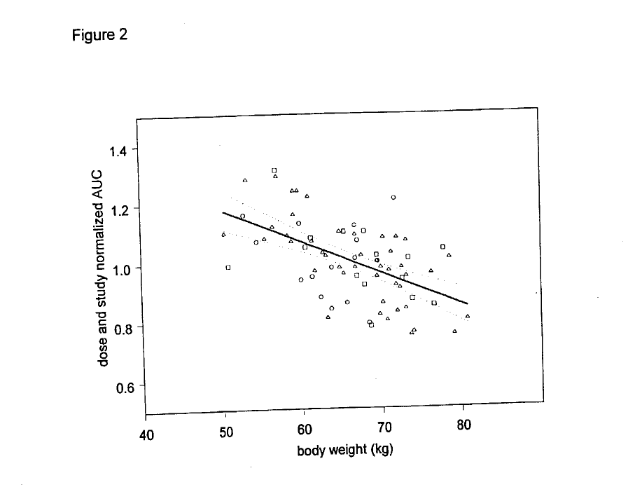

In all these trials A, B and C, blood samples were taken at regular intervals and the amount of ganirelix present in the blood was determined. Plots were prepared showing the amount of ganirelix as a function of time. This allowed the determination of the AUC. Next, the AUC was related to the body weight of the subjects. Results are indicated in FIG. 2.

A pooled analysis of the three pharmacokinetic studies has demonstrated that the clearance of ganirelix is positively related to body weight. This is expressed in a lower area under the curve (AUC) for subjects with a higher body weight (FIG. 2). Thus at the same dose level, individuals with a relatively high body weight will be exposed to lower levels of ganirelix, and individuals with a low body weight to relatively high levels.

As body weight is related to ganirelix levels it can be expected that body weight influences the clinical outcome.

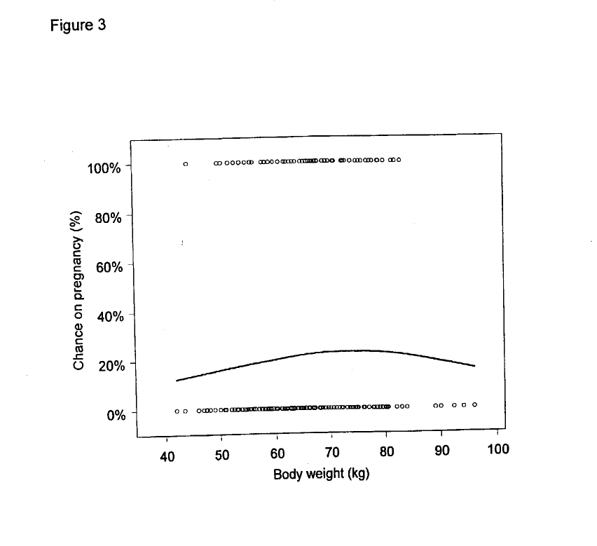

This hypothesis is supported by the results of a large Phase III efficacy study. At lower body weights a significant decrease in pregnancy rate was observed (see example 1 and FIG. 3). These subjects have been exposed to relatively higher levels of ganirelix. The pregnancy rate therefore could be optimized in these individuals by adjustment of the dose, according to their body weights.

### Example 4

Dose Finding

Pharmaco-statistical models have been set up to describe the influence of body weight on the effectiveness of ganirelix for both the prevention of LH-rises and pregnancy outcome. Spline functions have been applied to give the best and assumptionless mathematical description of the available data. Using these models optimal doses with respect to the prevention of LH-rises and pregnancy have been determined for different body weights.

TABLE 2
Relationship between body weight and optimal dose

| Weight (kg) | Optimal dose (μg) |
|---|---|
| <50 | <128 |
| 50-55 | 128 |
| 55-60 | 158 |
| 60-65 | 185 |
| 65-70 | 211 |
| 70-75 | 238 |
| 75-80 | 264 |
| >=80 | >264 |

Results are indicated in Table 2. Linear regression shows that these dosages can be given by the formula: (5.5*BW−166)±7% wherein BW is the body weight in kg.

1. In the treatment of female patients undergoing controlled ovarian hyperstimulation comprising administration of exogenous FSH and a GnRH antagonist, the improvement comprising administering said antagonist in an amount depending on the body weight of the patient wherein the relationship between body weight (BW in kg) and GnRH antagonist dosage (in micrograms) is defined by the Formula: (5.5*BW−166)±7% (Formula I).

2. Treatment according to claim 1 wherein said GnRH antagonist is ganirelix.

3. A cartridge containing a sterile liquid GnRH antagonist containing formulation in an amount so as to accurately administer to a patient an amount according to Formula 1 of claim 1 or 2.

4. The cartridge of claim 3 wherein the GnRH antagonist is ganirelix.

5. A device for administration of an adjustable sterile liquid GnRH antagonist comprising a cartridge according to claim 3 or 4.

6. Use of GnRH antagonist for the manufacture of a medicament to prevent a premature LH surge in female patients undergoing controlled ovarian hyperstimulation, the method of controlling ovarian hyperstimulation comprising administering to the patient a GnRH antagonist in an amount depending on the body weight of the patient wherein the relationship between body weight (BW in kg) and GnRH antagonist dosage (in micrograms) is defined by the Formula: (5.5*BW−166)±7%.

7. Use of GnRH antagonist according to claim 6 wherein said GnRH antagonist is ganirelix.

8. A kit for use in controlled ovarian hyperstimulation in female patients comprising a GnRH antagonist in a dosage form and quantity so as to administer accurately in an amount according to Formula I of claim 1 and a frequency effective to prevent a premature LH surge; FSH in a dosage form and quantity so as to administer in an amount and frequency effective to stimulate growth of follicles; and LH/hCG or a GnRH agonist in an dosage form and quantity so as to administer in an amount and frequency effective to induce ovulation.

9. Kit according to claim 8 wherein the antagonist is ganirelix.

10. Kit according to claims 8 or 9 wherein the antagonist is packaged in a cartridge according to claim 4.

11. Kit according to claims 9-10 comprising a device for administration of an adjustable sterile liquid amount of the antagonist.
